(19) United States
(12) Patent Application Publication
(10) Pub. No.: US 2016/0348085 A1
(43) Pub. Date: Dec. 1, 2016

(54) RECOMBINANT L-ASPARAGINASE FROM ZYMOMONAS

(71) Applicant: COPPE/UFRJ, INSTITUTO ALBERTO LUIZ COIMBRA DE PÓS- GRADUAÇÃO E PESQUISA DE ENGENHARRIA, Rio de Janeiro (BR)

(72) Inventors: Tito Livio Moitinho ALVES, Rio de Janeiro (BR); Karen EISNFELDT, Rio de Janeiro (BR); Isis Cavalcante BAPTISTA, Rio de Janeiro (BR); Rodrigo Volcan ALMEIDA, Rio de Janeiro (BR); Elaine Sobral DA COSTA, Rio de Janeiro (BR); Maria Cecilia Menks RIBEIRO, Rio de Janeiro (BR); Marcelo Greradin LAND, Rio de Janeiro (BR); Ariane Leites LARENTIS, Rio de Janeiro (BR)

(73) Assignee: COPPE/UFRJ, INSTITUTO ALBERTO LUIZ COIMBRA DE PÓS- GRADUAÇÃO E PESQUISA DE ENGENHARRIA, Rio de Janeiro (BR)

(21) Appl. No.: 15/110,697

(22) PCT Filed: Dec. 29, 2014
(86) PCT No.: PCT/BR2014/000455

§ 371 Date: Jul. 8, 2016

(30) Foreign Application Priority Data
Jan. 10, 2014 (BR) 102014000585-4

(51) Int. Cl. C12N 9/82

(52) U.S. Cl. CPC C12N 9/82; C12Y 305/01001; A61K 38/00

(57) ABSTRACT

The present invention relates to the construction and optimization of synthetic genes from the Zymomonas mobilis L-asparaginase gene, the method for cloning same and expression thereof in Escherichia coli. The purpose of the production of said enzyme is for producing high levels of a novel L-asparaginase that can be used in L-asparaginase-based pharmaceutical compositions for treating cancer, tumours and diseases involving cell proliferation, as well as for other medical applications.

### CROSS-REFERENCE TO RELATED APPLICATIONS

This application is the National Stage entry of International Application No. PCT/BR2014/000455, filed Dec. 29, 2014, which claims benefit of Brazilian Priority Application to BR 102014000585-4, filed Jan. 10, 2014, both of which are incorporated in their entirety by reference thereto.

### FIELD OF THE INVENTION

The present invention refers to the construction and optimization of synthetic L-asparaginase gene Zymomonas sp., preferably Zymomonas mobilis and the construction of plasmids for expression of intracellular and extracellular enzyme in bacteria and yeast. The invention relates to the production of this enzyme in submerged cultures, for use in pharmaceutical preparations L-asparaginase base used in the treatment of cancer, tumors, diseases of cell proliferation, as well as other medical applications.

### BACKGROUND OF INVENTION

For the treatment of a variety of lymphoproliferative disorders and lymphomas, particularly for acute lymphoblastic leukemia (ALL), the enzyme L-asparaginase (L-asparagine amino hydrolase, EC 3.5.1.1) is used as the chemotherapeutic agent (Narta et al. Critical Reviews in Oncology/Hematology 61, 208-221, 2007. Verma et al Critical Reviews in Biotechnology 27, 45-62, 2007). The antineoplastic activity of L-asparaginase occurs causes the depletion of exogenous supply of L-asparagine for the cells, since the malignant cells synthesize L-asparagine slowly in comparison with its need and rely on an exogenous supply of this amino acid. On the other hand, normal cells can normally synthesize the amino acid, and therefore are not damaged by the use of L-asparaginase (Lee et al Applied Biochemistry and Biotechnology 22 (1): 1-11, 1989; Duval et al Blood. 99 (8), 2734-2739, 2002; Kotzia Labrou and Journal of Biotechnology 119, 309-323, 2005 Rizzari et al Current Opinion in Oncology 25, S1-S9, 2013).

On the market are available formulations of native enzyme obtained from the microorganisms Escherichia coli and Erwinia chrysanthemi L-asparaginase of E. coli conjugated to polyethylene glycol (PEG) (Kurtzberg. In: Holland-Frei Cancer Medicine, 5th Ed., Hamilton Publisher (ON): B C Decker, 2000 Van Den Berg Leukemia & Lymphoma, 52 (2), 168-178, 2011). During the treatment, the main problem in the use of L-asparaginase is the clinical hypersensitivity developed by patients treated with the unmodified forms of the enzyme. Studies with L-asparaginases from E. coli and Erwinia chrysanthemi showed that both enzymes have high immunogenicity (Narta et al. Critical s Revie in Oncology/Hematology. 61, 208-221, 2007). However, these enzymes (from E. coli and Erwinia chrysanthemi) serve as an alternative if the patient develops sensitivity to one of them (Kotzia and Labrou. Journal of Biotechnology. 119, 309-323, 2005 Rizzari et al. Current Opinion in Oncology. 25, S1-S9, 2013). Another alternative to the problem of hypersensitivity is to use another type of L-asparaginase (obtained from a different micro-organism or conjugated to polyethylene glycol), therefore it is very important to search for new sources of L-asparaginase which have antileukemic activity. A new source of L-asparaginase enzyme is the Zymomonas mobilis bacteria.

The Zymomonas mobilis bacterium is reported to have therapeutic properties. There are reports of therapeutic applications of Zymomonas cultures in cases of bacterial enterocolitis and gynecological infections (Wanik and Silva Antibiotics Institute Journal 11, 69-71, 1971; Lopes et al Journal of Antibiotics Institute 20, 69-77, 1980).

However, low productivity of L-asparaginase enzyme by Z. mobilis becomes an obstacle to the study and production of the enzyme from this organism. This obstacle can be overcome by the production of the recombinant enzyme at high concentrations, using genetic engineering.

To meet the industrial demand, proteins of interest can be produced in large quantities using genetic engineering (Demain and Vaishnav. Biotechnology Advances. 27, 297-306, 2009). The bacterium Escherichia coli is the most widely used expression systems for recombinant proteins, including on a commercial scale (Baneyx Current Opinion in Biotechnology 10, 411-421, 1999; TERPE Applied Microbiology and Biotechnology 72, 211-222, 2006.) This is because, besides offering several advantages such as the ability to grow rapidly at high cell concentrations and cheap substrates, it has well characterized genetic and there are several vectors and commercially available mutant strains (Baneyx. Current Opinion in Biotechnology. 10, 411-421, 1999; Page and PETI Protein Expression and Purification 51: 1-10, 2007). In addition, cells from E. coli have ability to accumulate over 80% of its dry weight in recombinant protein (Demain and Vaishnav. Biotechnology Advances. 27, 297-306, 2009).

### PRIOR ART

The gene manipulation technique has been widely used to develop production systems of the different types of proteins with diverse purposes. The book Molecular Cloning: A Laboratory Manual (Sambrook J, Fritsch E F, Maniatis T. 1989. Molecular Cloning: A Laboratory Manual, 2nd edition Cold Spring Harbor Laboratory Press, New York) describes in detail the process of genetic manipulation for obtaining recombinant cells.

The bacterium Escherichia coli is the most widely used expression systems for recombinant proteins, including on a commercial scale (Baneyx Current Opinion in Biotechnology 10, 411-421, 1999; TERPE Applied Microbiology and Biotechnology 72, 211-222, 2006.). Several patent documents use E. coli as the micro-organism for expression of recombinant proteins, including L-asparaginases. As well as some patent documents produce recombinant L-asparaginases from different microorganisms in hosts such as Escherichia coli and yeasts. However, there are no documents reporting the cloning and expression of recombinant L-asparaginase Zymomonas sp. in any micro-organism.

In the patent document WO2003/0186380 are described recombinant methods for producing a polypeptide with at least 70% identity with the sequence of amino acids L-asparaginase ATCC6051A of Bacillus subtilis

There are documents that report inventions of modified forms of L-asparaginase. The invention US2012/0100121 refers to a chemically modified form of the enzyme from Erwinia L-asparaginase, conjugated with a molecule of polyethylene glycol, particularly with molecular weight less than or equal to 5000 Da, and its clinical use. The document US20100284982 describes compositions for the transporting of L-asparaginase through the cell membrane of erythrocytes, the preparation process of these compositions and the method of its clinical use.

In the patent document WO2008/011234A2 it is described the cloning and production of L-asparaginase II from Escherichia coli using Escherichia coli as a host, resulting in a recombinant strain of E. coli with a vector extrachromosomal and chromosomal DNA coding for the same L-asparaginase II. The document WO2010/015264A1 relates to cloning, production and characterization of the L-asparaginase enzyme of Helicobacter pylori CCUG 17874 using Escherichia coli as a host. As for the Brazilian patent application PI0406168-3 it relates to production of L-asparaginase Saccharomyces cerevisiae by cloning the gene encoding such enzyme in a methylotrophic yeast. The patent application PI 0404952-7, which has the same applicant of the present one, refers to the obtaining process of the native L-asparaginase enzyme of Zymomonas mobilis, by culture of wild Zymomonas mobilis for production of the enzyme, in which activity was obtained 37.79 IU/g in 33 hours of cell culture.

### SUMMARY OF THE INVENTION

The present invention reports the construction and optimization of synthetic L-asparaginase of Zymomonas mobilis gene as well as the construction of plasmids for expression of intracellular and extracellular enzyme in Escherichia coli, the subsequent insertion of these plasmids into Escherichia coli and the production of enzyme in submerged fermentation. The development of this recombinant enzyme aim for its use in pharmaceutical compositions intended for use in medical applications, such as in the treatment of cancer, tumors and diseases in which cell proliferation is involved, providing an alternative to existing drugs.

The products developed in this invention represent a new way to treat cancer, tumors and diseases in which cell proliferation is involved. In addition, the developed techniques provide a high production output and productivity, which is reflected in lower cost of the final product.

### BRIEF DESCRIPTION OF THE FIGURES

FIG. 1 shows the alignment using the CLUSTAL 2.0.1 Multiple Sequence Alignment software of the 1-asparaginase gene (original) with L-asparaginase gene (optimized). The underlined nucleotides in bold represent those that are part of the signal peptide of the original gene that were withdrawn from the optimized gene. The underlined nucleotides refers to the histidine tail and enterokinase cleavage site added to the optimized gene.

FIG. 2 represents the electrophoretic pattern and digestion of plasmids extracted from three clones of Escherichia coli BL21(DE3)/pET26b/asparaginase, digested with the restriction enzyme (Xho\). M Marker 1 kb GeneRuler™ DNA Ladder (Fermentas), i-vector pET26b/intact asparaginase, d-vector pET26b/asparaginase digested with the Xhol enzyme. The arrow indicates the 6368 bp fragment corresponding to the linearized pET26b/asparaginase vector.

FIG. 3 represents the electrophoretic pattern and digesting of the plasmids extracted of 3 clones of Escherichia coli BL21 (DE3)/pET28a/asparaginase, digested with the restriction enzyme (Xho\). M GeneRuler™ DNA Marker 1 kb Ladder (Fermentas), 1-intact pET28a/asparaginase vector, 2-pET28a/asparaginase vector digested with the enzyme Xhol. The arrow indicates the 6301 bp fragment corresponding to the linearized pET28a/asparaginase vector.

FIG. 4 shows the production chart of extracellular L-asparaginase by the Escherichia coli BL21(DE3)/pET26b/asparaginase bacteria and cell growth in the bioreactor using LB medium. The Y-axis represents the cell concentration (g/L). The Z axis represents the enzyme activity (IU/ml). The X axis represents time (minutes). ▪—Enzyme Activity (IU/ml); ♦—Cell Concentration (g/L). The bars represent standard deviations.

FIG. 5 shows the production chart of intracellular L-asparaginase by the Escherichia coli BL21 (DE3)/pET28a/asparaginase bacteria and cell growth in the bioreactor using LB medium. The Y-axis represents the cell concentration (g/L). The Z axis represents the enzyme activity (IU/ml). The X axis represents time (minutes). ▪—Enzyme Activity (IU/ml); ♦—Cell Concentration (g/L). The bars represent standard deviations.

FIG. 6 shows the specific activity graphic of intracellular L-asparaginase (using Escherichia coli BL21(DE3)/pET28a/asparaginase) and extracellular (using Escherichia coli BL21(DE3)/pET26b/asparaginase) produced in the bioreactor using LB medium. The Y-axis represents the specific activity of the L-asparaginase (IU/mg). The X axis represents time (minutes). ▪—intracellular L-asparaginase (IU/mg); ♦—extracellular L-asparaginase (IU/mg).

FIG. 7 shows the cell viability analysis chart by flow cytometry, through staining with propidium iodide (PI) in primary sample of bone marrow exposed to various concentrations of L-asparaginase recombinant of Z mobilis. The Y-axis represents the lateral spreading of the cells. The X-axis represents the fluorescence intensity per IP. 1—control, 2—1.0 IU/mL, 3—0.5 IU/mL, 4—0.1 UI/mL, 5—0.05 UI/ml 0.025, 6—IU/ml.

### DETAILED DESCRIPTION OF THE INVENTION

Two vectors were constructed containing the gene that encodes the enzyme L-asparaginase from Zymomonas mobilis, a vector for the intracellular enzyme expression and another vector for the extracellular enzyme expression. In both vectors the gene encoding L-asparaginase enzyme was based on the sequence of Zymomonas mobilis subsp. mobilis ZM4 deposited in genbank database with 3,189,240 ID reference to the gene and YP_163418.1 to the amino acid sequence, which can be accessed at http://www.ncbt.nlm.nih.gov address. By inserting these vectors in Escherichia coli were obtained two distinct clones, both able to express the recombinant L-asparaginase protein.

The invention will be detailed by examples in the following text. The invention is not limited by the examples presented below, those being for illustrative purposes, for a better understanding of the invention reported in this document.

### Example 1

### Synthesis of the L-Asparaginase Gene

The gene type II of Zymomonas mobilis of the L-asparaginase gene was chemically synthesized by the company Epoch Life Science Inc. The design of the gene was performed using the sequence of the 1 01 nucleotides of the strain Zymomonas mobilis subsp. mobilis ZM4 gene deposited in the GenBank database by the 3189240 ID reference and illustrated herein as SEQ ID NO: 2, it sequence encodes a protein of 366 amino acids. The gene was synthesized based on the sequence of the L-asparaginase gene of Zymomonas mobilis subsp. mobilis ZM4 noted above, removing the nucleotides encoding the first 29 amino acids of the sequence, as those are part of a signal sequence (signal peptide). To that sequence was added a nucleotide sequence encoding six histidine, allowing the expression of the fused protein with a histidine tag at its N-terminus far end. Were also added nucleotides encoding the amino acid sequence: Asp-Asp-Asp-Asp-Lys, a cleavage site of the enterokinase enzyme for later removal of the histidine tag. The codons used in the synthetic gene were optimized by the most frequent codons used by Escherichia coli. The synthetic gene, flanked by restriction enzymes Ncol and Xhol, was inserted in the pET26b vector (Novagen), which has a signal sequence (signal peptide), pelB (Erwinia carotovora native), to export the protein to the space periplasmic, where it will be secreted into the culture medium. This same synthetic gene, flanked by the restriction enzymes Nco\ and Xho\, was also inserted into pET28a vector (Novagen) to express the protein in the cytoplasm. The sequence of the optimized and synthesized gene is shown in SEQ ID NO: 1. The sequence of the pET26b plasmid containing the optimized gene is shown in SEQ ID NO: 3 and the sequence of the pET28a plasmid containing the optimized gene is shown in SEQ ID NO: 4. The optimized sequence SEQ ID NO: 1, has a translation represented in SEQ ID NO: 5.

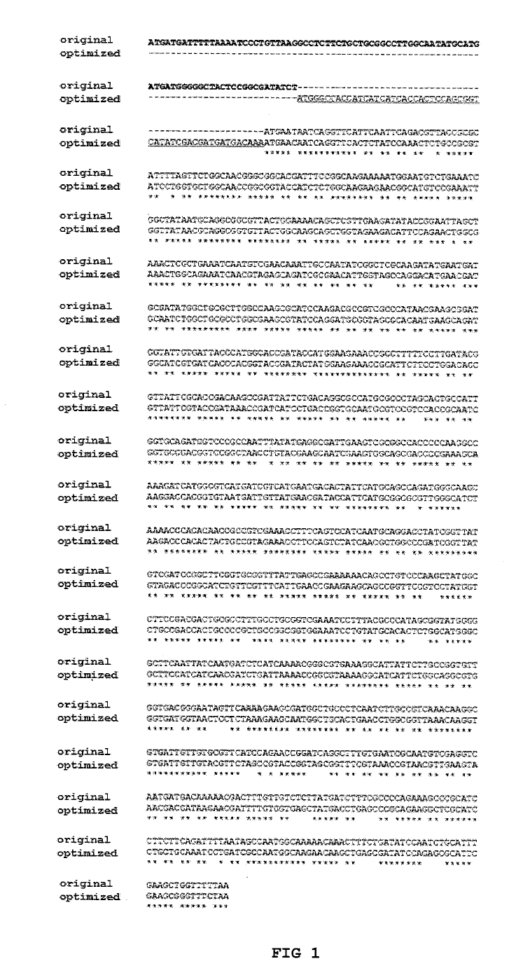

The alignment of the original L-asparaginase gene (SEQ ID NO: 2) with L-asparaginase optimized gene (SEQ ID NO: 1) is shown in FIG. 1.

### Example 2

### Transformation into Escherichia coli

Eletrocompetent E. coli DH5a and E. coli BL21 (DE3) cells were used as hosts for the plasmids pET26b/asparaginase and pET28a/asparaginase. The insertion of those plasmids in the cells was performed by electroporation. For the electroporation, the plasmids and 100 ul of eletrocompetent E. coli were gently mixed to avoid the formation of bubbles. Each mixture was transferred to a cuvette of 0.2 cm thick between the electrodes, and then subjected to an electric discharge for about 5 ms, with a voltage of 2.5 kV, capacitance of 25 pF and resistance of 200Ω at a eletroporator Gene Pulser® II (Bio-Rad). After the electric shock, were quickly added 1 mL of sterile LB medium and the mixture was incubated at 37° C. under agitation at 200 rpm for 1 hour. After this period, 200 pL of the mixture were spread on LB agar plate containing 50 pg/ml of canamictna. The plates were incubated at 37° C. for 16 hours. This procedure was first carried out with DH5et E. coli strain and after confirmation of the clones, the plasmids were extracted and transformed into the expression strain E. coli BL21 (DE3).

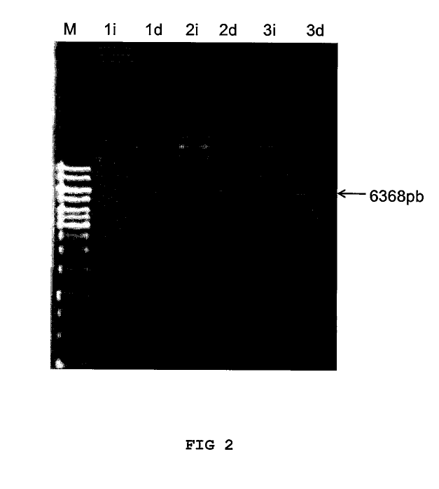

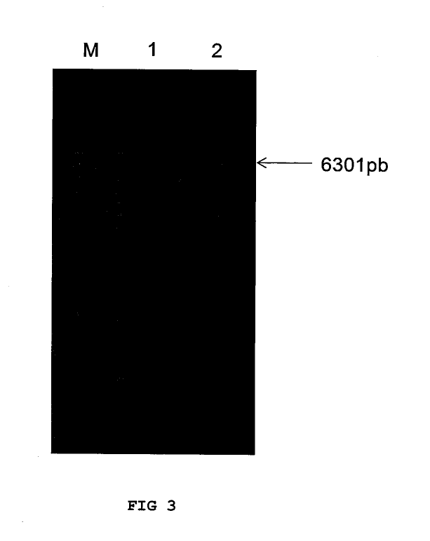

The selection of clones transformed with the plasmids was made by spreading the cells on LB agar with selective pressure (use of the kanamycin antibiotic). To make the selection, some colonies present on the plate were selected, and each inoculated into 10 mL of liquid LB medium (5 g/L yeast extract, 10 g/L of tryptone, 10 g/L of NaCl) with 50 pg/ml of kanamycin and 1% of glucose and incubated at 37° C. and 200 rpm for 16 hours. From these crops were made glycerol stocks (called Mother Stock), stored at −8Q ° C., and the plasmid extractions for the confirmation of clones (electrophoretic and digestion default). The electrophoretic and digestion pattern of the clones of BL21 (DE3)/pET26b/asparaginase Escherichia coli and BL21 (DE3)/pET28a/asparaginase Escherichia coli are shown in FIGS. 2 and 3, respectively. After confirming the clones, glycerol stocks called Working Lot were made. For this purpose, were inoculated 10 pL from the Mother Stock in 10 ml of LB medium with 1% of glucose and 50 pg/ml of kanamycin and incubated at 37° C. under agitation of 200 rpm, until they reached Absorbance (600 nm) of approximately 1 0. Several aliquots with 500 pL of the cultivation and 500 pL of 50% sterile glycerol were prepared. The aliquots (Working Lot) were stored at −80° C.

### Example 3

### Analysis of L-Asparaginase Expression in Shake Flasks

To analyze the expression of the L-asparaginase enzyme cultures were performed in triplicate for each recombinant bacteria (E. coli BL21 (DE3)/p T28a/asparaginase and E. coli BL21 (DE3)/pET26b/asparaginase) in shake flasks. Therefore, each pre-inoculum was prepared by inoculating 10 uL of the Working Lot of the recombinant bacteria E. coli BL21 (DE3)/pET28a/asparaginase and E. coli BL21 (DE3)/pET26b/asparaginase in 10 ml of LB medium with 1% of glucose and 50 pg/ml of kanamycin. The pre-inoculum of each bacterium was incubated for 16 hours at 37° C. and 200 rpm in 50 ml shake flasks. After 16 hours, the inoculum of each bacterium was prepared in 50 ml of LB medium with 1% of glucose and 50 pg/ml of kanamycin were inoculated with 1 ml of the pre-inoculum in 250 mL flasks. The cultures were incubated at 37° C. and 200 rpm until they reached the exponential phase of growth (approximately 1.0 of 600 nm Absorbance). At this point, the protein expression was induced by 0.55 mM IPTG (isopropyl-PD-thiogalactopyranoside) for 4 hours. At the end of the four hours of expression the cell growth of crops was assessed by absorbance 600 nm (Abs600 nm). Samples of 1 ml were taken from crops of E. coli BL21 (DE3)/pET28a/asparaginase and 10 ml of crops of E. coli BL21 (DE3)/pET26b/asparaginase to examine the L-asparaginase expression by enzyme activity. To evaluate the expression of the protein obtained from the E. coli BL21 (DE3)/pET28a/asparaginase the cell pellets obtained from 1 ml of samples taken from cultures were used after 4 hours of expression. The precipitates were resuspended by adding 1 ml of 0.02 M sodium phosphate buffer, pH 7.3, and then subjected to ultrasound for five cycles of 10 seconds with 30% amplitude in a sonicator. These samples were then used for analysis of enzyme activity. To evaluate the expression of the protein obtained from the E. coli BL21 (S03)/pET26b/asparaginase were used 10 ml of the cell-free culture medium removed from the crops after 4 hours of expression. These samples of culture medium were concentrated 10 times and the medium was replaced by 0.02 M pH 7.3 sodium phosphate buffer, using ultrafiltration units with a 10 kDa membrane (Amicon Ultra-15, illipore) and centrifugations at 4000 g for 30 minutes. The samples were then used for analysis of enzyme activity. For the analysis of enzymatic activity, 50 uL of the sample were added to 50 pL of 5 g/L asparagine. This reaction was incubated at 37° C. for 30 minutes. Immediately after this time, 40 ul of this reaction were added to 10 pL of 1.5 M TCA (trichloroacetic acid). To the resulting 50 pL were added 2 ml of a reactant compound of a mixture of sodium salicylate, sodium nitroprusside and disodium EDTA and the mixture is stirred. In the sequence is added 2 ml of a second reagent compound of sodium hypochlorite and sodium hydroxide (Tabacco et al. Clinical Chemistry. 25 (2), 336-337, 1979). This is because the ammonium ions in the presence of these reagents form a compound chromogen blue-green. The mixture is incubated at 37° C. for 5 minutes. The sample is then subjected to reading absorbance at a wavelength of 600 nm. From a standard curve, were determined the concentrations of ammonium ion released during the enzymatic reaction. One international unit of asparaginase (IU) was defined as the quantity of enzyme that is capable of releasing 1μιηoI ammonia per minute at 37° C. and pH 7.3. The results obtained in these experiments, both cell growth and the expression of L-asparaginase, can be seen in Table 1 and 2. The enzyme activity results are presented as IU/ml of culture medium.

TABLE 1

| | Average deviation | Standard |
|---|---|---|
| Recombinant bacteria | Concentration (g/L) | Deviation |
| E. coli | 0.4565 | 0.018 |
| BL21(DE3)/pET28a/asparaginase | | |
| E. coli | 0.8986 | 0.011 |
| BL21(DE3)/pET26b/asparaginase | | |

TABLE 2

| | | Standard |
|---|---|---|
| Recombinant bacteria | UI/mL (average) | Deviation |
| E. coli | 7.630 | 0.844 |
| BL21(DE3)/pET28a/asparaginase | | |
| E. coli | 0.189 | 0.038 |
| BL21(DE3)/pET26b/asparaginase | | |

The results from these experiments show that L-asparaginase gene from Zymomonas mobilis was expressed in both recombinant bacteria produced. The L-asparaginase has been secreted into the culture medium when the bacterium E. coli recombinant BL21 (DE3)/pET26b/asparaginase was used for the expression, while the other recombinant bacteria produced in this study (E. Coli BL21 (DE3)/pET28a/asparaginase) was used, there was expression of the protein in the cytoplasm.

### Example 4

### Production of L-Asparaginase in Bioreactor

The L-asparaginase enzyme was produced in bioreactors. A bioreactor was used for the cultivation of recombinant bacteria E. coli BL21 (DE3)/pET28a/asparaginase and enzyme production in the cytoplasm of the bacterium, another bioreactor was used for the cultivation of recombinant E. coli BL21 (DE3)/pET26b/asparaginase and enzyme production in the culture medium. Two pre-inoculums were made, one with E. coli BL21 (DE3)/pET28a/asparaginase and the other with E. coli BL21 (DE3)/pET26b/asparaginase. To this end, 10 ul of the bacteria working lot were inoculated into 50 ml of LB medium with 1% glucose and 50 pg/ml kanamycin in a 250 ml flasks. The pre-inoculum of each bacteria was incubated for 16 hours at 37° C. with agitation of 200 rpm. After this time were used 8 ml of the pre-inoculum to inoculate a bioreactor containing 400 ml of LB medium with 1% glucose and 50 pg/ml kanamycin. The cultures were conducted at 37° C. under agitation of 200 rpm-800 rpm. The pH of culture was maintained at 7.0. When the cell growth reached 600 nm absorbance of approximately 2.0, the protein expression was induced by 0.55 mM IPTG for 4 hours. Samples were taken hourly to evaluate cell growth through 600 nm absorbance measurements and after induction the samples were also taken to assess the expression of protein by enzyme activity analysis. The enzyme activity analyzes were conducted as described above in the analysis of samples taken from experiments in shake flasks.

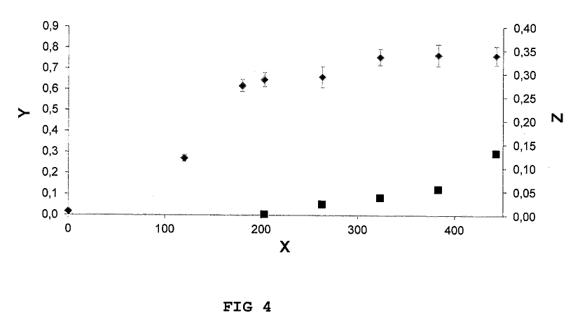

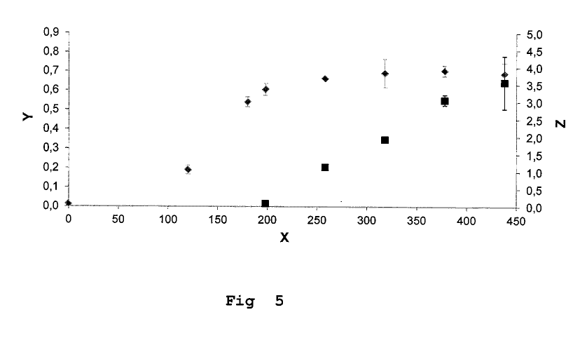

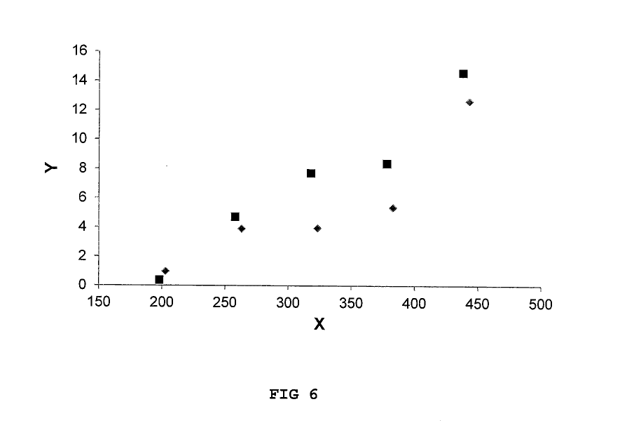

In the cultivation using E. coli strain BL21 (DE3)/pET26b/asparaginase with extracellular L-asparaginase production was obtained the activity of 0.132 IU/ml culture medium and 172.7 IU/g cell after 4 hours of induction expression. The results of cell growth and protein production of this culture are shown in FIG. 4. In the cultivation using E. coli strain BL21 (DE3)/pET28a/asparaginase with production of L-asparaginase was obtained by intracellular activity of 3.57 IU/ml of culture medium or 5185 IU/g cell after 4 hours of induction of expression. The results of cell growth and protein production of this cultivation are shown in FIG. 5. It is also presented the values of specific activity of each crop (IU/mg total protein), the extracellular and intracellular L-asparaginase in FIG. 6.

It is noteworthy that the production amounts of L-asparaginase presented in this patent application are higher than those presented by the native micro-organism of this L-asparaginase (Zymomonas mobilis), according to the culture in liquid medium in shake flasks, described in the brazilian patent application PI 0404952-7, which has the same applicant of the present one and in which activity was obtained 37.79 IU/g of cells in 33 hours of cultivation. It is clear that after our intervention presented in this patent application, it was possible to obtain approximately 135 times more IU/g cell, at a time four times lower.

### Example 5

### Cytotoxicity Analysis of L-Asparaginase

In those experiments we used the Z. mobilis recombinant L-asparaginase enzyme produced by recombinant bacterium E. coli BL21 (DE3)/pET26b/asparaginase described in this patent application. To verify the in vitro cytotoxicity of the L-asparaginase on the leukemic cells of a patient with acute lymphoblastic leukemia, it was used a primary sample of bone marrow obtained at diagnosis. Mononuclear cells, from human bone marrow sample from a patient 4 years old with acute lymphoblastic leukemia, were obtained at the time of their diagnosis, isolated by density gradient Ficoll-Paque. For the treatment with recombinant L-asparaginase, the cells were washed with RPMI 1640 culture medium, centrifuged and resuspended in L-glutamine-free RPMI 1640 culture medium, supplemented with 20% fetal bovine serum, 2 mmol/LL glutamine, 100 IU/ml penicillin and 100 vglmL of streptomycin. The cells were inoculated into tissue culture dishes of wells, 10⁶ cells/well in 1 ml of medium with increasing concentrations of the recombinant L-asparaginase. In the control, the enzyme was not added. Both were incubated at 37° C. in 5% CO2 humid atmosphere for 48 hours. After this period, cell viability was done by flow cytometry analysis. To assess viability, cells were marked with propidium iodide (PI). Before the acquisition, a compound pattern of two types of fluorescent microspheres was added to the samples. The data acquisition was done using the FACSDIVA software on a FACSCanto II flow cytometer (Becton Dickinson, Sans Jose, Calif., USA). The data analysis was performed using the Infinicyte program (Cytognos S L, Salamanca, Spain).

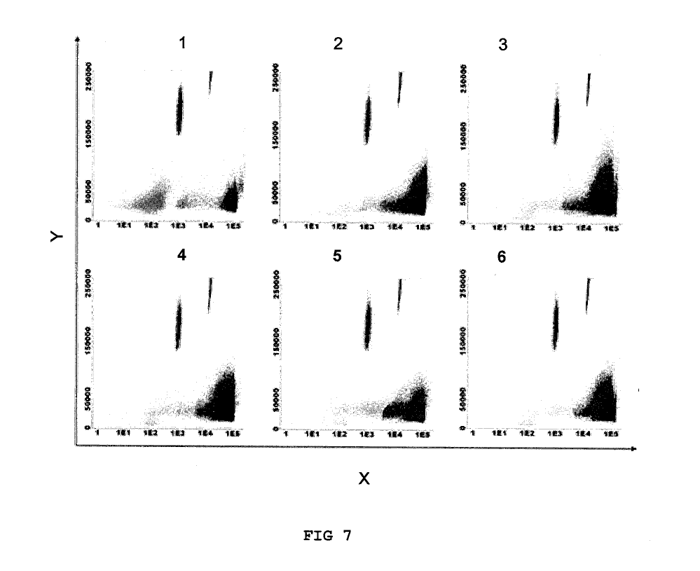

The effect of the different concentrations tested on cell viability is shown by the marking of fluorophore propidium iodide (PI) in FIG. 7, wherein the dead cells are marked with PI and emit fluorescence. The number of events and the percentage of death are shown in Table 3. It was observed that the percentage of dead cells in the samples that received treatment was higher than the control for all tested concentrations of enzyme, reaching more than 80% of death in concentration 1.0 IU/ml, however reaching approximately 79% at a concentration of 0.1 IU/ml.

TABLE 3

| Concentration | Number of | Number of | | % dead |
|---|---|---|---|---|
| (UI/mL) | events | dead cells | % Microspheres | cells |
| 1.0 | 140.608 | 100.769 | 12.18 | 80.78 |
| 0.5 | 189.904 | 107.939 | 12.56 | 79.42 |
| 0.1 | 143.780 | 98.944 | 11.53 | 78.94 |
| 0.05 | 133.302 | 73.271 | 15.46 | 68.71 |
| 0.025 | 113.126 | 74.911 | 16.95 | 75.33 |
| 0.00* | 129.324 | 38.851 | 16.01 | 30.04 |

*Control

The description made until this point of the L-asparaginases production process by means of synthetic gene construction and its insertion in Escherichia coli, object of the present invention, should be regarded only as a possible or possible embodiments, and any particular characteristics therein introduced shall be construed as illustrative, aiming only to facilitate understanding. Thus, they can in no way be considered as limitation to the invention, which is limited to the scope of the claims that follow.

1- SYNTHETIC DEOXYRIBONUCLEIC ACIDS (DNAs), characterized by comprising the optimized nucleotide sequences that encode into L-asparaginases Zymomonas.

2- SYNTHETIC DEOXYRIBONUCLEIC ACIDS (DNAs) as in claim 1, characterized by encoding the L-asparaginases of Zymomonas mobilis.

3- SYNTHETIC DEOXYRIBONUCLEIC ACIDS (DNAs) as in claim 1, characterized by SEQ ID NO: 1.

4- EXPRESSION VECTORS IN BACTERIA characterized by SEQ ID NO: 3, SEQ ID NO: 4.

5- BACTERIA AND RECOMBINANT YEAST characterized by being transformed with the DNAs specified in claim 1.

6- RECOMBINANT BACTERIA characterized for being Escherichia coli containing the DNAs specified in claim 1,

7- RECOMBINANT YEAST characterized for being Pichia pastoris containing the DNA specified in claim 1.

8- FUSION PROTEIN, characterized by SEQ ID NO: 5 obtained from deoxyribonucleic acid according to claim 1, represented by SEQ ID NO 1.

9- PRODUCTION PROCESS OF L-ASPARAGINASES BY BACTERIA, characterized by comprising the construction of synthetic DNAs according to claim 1, its insertion in the bacteria Escherichia coli, so that it passes to produce L-asparaginases in high levels of expression and high productivity

10- PRODUCTION PROCESS OF L-ASPARAGINASES BY YEAST, characterized by comprising the construction of synthetic DNAs according to claim 1, its insertion into the Pichia pastoris yeast, so that it passes to produce L-asparaginases in high expression levels and high productivity.

11. PRODUCING PROCESS OF L-ASPARAGINASES BY GENETIC MODIFICATION OF BACTERIAS OR YEASTS, according to claim 1, characterized by cultivation conducted between 15 and 37° C. under agitation from 200 to 800 rpm, and its pH maintained between 5.0 and 7.0.

12. COMPOSITION characterized by comprising L-asparaginase obtained from processes according to claims 9 and 10, composed of L-asparaginase with or without excipients such as mannitol, sorbitol, sodium chloride, dextrose.

13. USE OF L-ASPARAGINASES for preparing the base formulations L-asparaginase used to treat cancer, tumors, diseases of cell proliferation, as well as other medical applications.

14. METHOD OF TREATING cancer using the composition according to claim 12.


SEQUENCE LISTING (5 sequences)

SEQ ID NO: 1 - DNA, 1068 bp, Artificial Sequence
  optimized L-asparaginase gene
atgggccaccatcatcatcaccactccagcggtcatatcgacgatgatgacaaaatgaacaatcaggttcactctatccaaactctgccgcgtatcctggtgctggcaaccggcggtaccatctctggcaagaagaacggcatgtccgaaattggttataacgcaggcggtgttactggcaagcagctggtagaagacattccagaactggcgaaactggcagaaatcaacgtagagcagatcgcgaacattggtagccaggacatgaacgatgcaatctggctgcgcctggcgaagcgtatccaggatgcggtagcgcacaatgaagcagatggcatcgtgatcacccacggtaccgatactatggaagaaaccgcattcttcctggacaccgttattcgtaccgataaaccgatcatcctgaccggtgcaatgcgtccgtccaccgcaatcggtgccgacggtccggctaacctgtacgaagcaatcgaagtggcagcgaccccgaaagcaaaggaccacggtgtaatgattgttatgaacgataccattcatgcggcgcgttgggcatctaagacccacactactgccgtagaaaccttccagtctatcaacgctggcccgatcggttatgtagacccggcatctgttcgtttcattgaaccgaagaagcagccggttccgtcctatggtctgccgaccactgccccgctgccggcggtggaaatcctgtatgcacactctggcatgggcgcttccatcatcaacgatctgattaaaaccggcgtaaaaggcatcattctggcaggcgtgggtgatggtaactcctctaaagaagcaatggctgcactgaacctggcggttaaacaaggtgtgattgttgtacgttctagccgtaccggtagcggtttcgtaaaccgtaacgttgaagtaaacgacgataagaacgattttgtggtgagctatgacctgagcccgcagaaggctcgcatcctgctgcaaatcctgatcgccaatggcaagaacaagctgagcgatatccagagcgcattcgaagcgggtttctaa

SEQ ID NO: 2 - DNA, 1100 bp, Zymomonas mobilis
atgatgatttttaaaatccctgttaaggcctcttctgctgcggccttggcaatatgcatgatgatgggggctactccggcgatatctatgaataatcaggttcattcaattcagacgttaccgcgcattttagttctggcaacgggcggcacgatttccggcaagaaaaatggaatgtctgaaatcggctataatgcaggcggcgttactggaaaacagctcgttgaagatataccggaattagctaaactcgctgaaatcaatgtcgaacaaattgccaatatcggctcgcaagatatgaatgatgcgatatggctgcgcttggccaagcgcatccaagacgccgtcgcccataacgaagcggatggtattgtgattacccatggcaccgataccatggaagaaaccgcctttttccttgatacggttattcgcaccgacaagccgattattctgacaggcgccatgcgccctagcactgccattggtgcagatggtcccgccaatttatatgaggcgattgaagtcgcggccacccccaaggccaaagatcatggcgtcatgatcgtcatgaatgacactattcatgcagccagatgggcaagcaaaacccacacaaccgccgtcgaaacctttcagtccatcaatgcaggacctatcggttatgtcgatccggcttcggtgcggtttattgagccgaaaaaacagcctgtcccaagctatggccttccgacgactgcgcctttgcctgcggtcgaaatcctttacgcccatagcggtatgggggcttcaattatcaatgatctcatcaaaacgggcgtgaaaggcattattcttgccggtgttggtgacgggaatagttcaaaagaagcgatggctgccctcaatcttgccgtcaaacaaggcgtgattgttgtgcgttcatccagaaccggatcaggctttgtgaatcgcaatgtcgaggtcaatgatgacaaaaacgactttgttgtctcttatgatctttcgccccagaaagcccgcatccttcttcagattttaatagccaatggcaaaaacaaactttctgatatccaatctgcatttgaagctggttttta

SEQ ID NO: 3 - DNA, 6364 bp, Artificial Sequence
  optimized pET26b/asparaginase sequence
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttagaaacccgcttcgaatgcgctctggatatcgctcagcttgttcttgccattggcgatcaggatttgcagcaggatgcgagccttctgcgggctcaggtcatagctcaccacaaaatcgttcttatcgtcgtttacttcaacgttacggtttacgaaaccgctaccggtacggctagaacgtacaacaatcacaccttgtttaaccgccaggttcagtgcagccattgcttctttagaggagttaccatcacccacgcctgccagaatgatgccttttacgccggttttaatcagatcgttgatgatggaagcgcccatgccagagtgtgcatacaggatttccaccgccggcagcggggcagtggtcggcagaccataggacggaaccggctgcttcttcggttcaatgaaacgaacagatgccgggtctacataaccgatcgggccagcgttgatagactggaaggtttctacggcagtagtgtgggtcttagatgcccaacgcgccgcatgaatggtatcgttcataacaatcattacaccgtggtcctttgctttcggggtcgctgccacttcgattgcttcgtacaggttagccggaccgtcggcaccgattgcggtggacggacgcattgcaccggtcaggatgatcggtttatcggtacgaataacggtgtccaggaagaatgcggtttcttccatagtatcggtaccgtgggtgatcacgatgccatctgcttcattgtgcgctaccgcatcctggatacgcttcgccaggcgcagccagattgcatcgttcatgtcctggctaccaatgttcgcgatctgctctacgttgatttctgccagtttcgccagttctggaatgtcttctaccagctgcttgccagtaacaccgcctgcgttataaccaatttcggacatgccgttcttcttgccagagatggtaccgccggttgccagcaccaggatacgcggcagagtttggatagagtgaacctgattgttcattttgtcatcatcgtcgatatgaccgctggagtggtgatgatgatggtggcccatggccatcgccggctgggcagcgaggagcagcagaccagcagcagcggtcggcagcaggtatttcatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccaaggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacggcagaaaaaaaggatctcaagaagatcctttgatcttttctacggggtctgacgctcagtggaacgaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 4 - DNA, 6301 bp, Artificial Sequence
  optimized pET28a/asparaginase sequence
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttagaaacccgcttcgaatgcgctctggatatcgctcagcttgttcttgccattggcgatcaggatttgcagcaggatgcgagccttctgcgggctcaggtcatagctcaccacaaaatcgttcttatcgtcgtttacttcaacgttacggtttacgaaaccgctaccggtacggctagaacgtacaacaatcacaccttgtttaaccgccaggttcagtgcagccattgcttctttagaggagttaccatcacccacgcctgccagaatgatgccttttacgccggttttaatcagatcgttgatgatggaagcgcccatgccagagtgtgcatacaggatttccaccgccggcagcggggcagtggtcggcagaccataggacggaaccggctgcttcttcggttcaatgaaacgaacagatgccgggtctacataaccgatcgggccagcgttgatagactggaaggtttctacggcagtagtgtgggtcttagatgcccaacgcgccgcatgaatggtatcgttcataacaatcattacaccgtggtcctttgctttcggggtcgctgccacttcgattgcttcgtacaggttagccggaccgtcggcaccgattgcggtggacggacgcattgcaccggtcaggatgatcggtttatcggtacgaataacggtgtccaggaagaatgcggtttcttccatagtatcggtaccgtgggtgatcacgatgccatctgcttcattgtgcgctaccgcatcctggatacgcttcgccaggcgcagccagattgcatcgttcatgtcctggctaccaatgttcgcgatctgctctacgttgatttctgccagtttcgccagttctggaatgtcttctaccagctgcttgccagtaacaccgcctgcgttataaccaatttcggacatgccgttcttcttgccagagatggtaccgccggttgccagcaccaggatacgcggcagagtttggatagagtgaacctgattgttcattttgtcatcatcgtcgatatgaccgctggagtggtgatgatgatggtggcccatggtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 5 - protein, 359 aa, Artificial Sequence
  protein translation of SEQ ID NO 1
MGHHHHHHSSGHIDDDDKMNNQVHIQTSLPRILAVLTGGTISGKGKNMSEIGYNAGGVTGKQLVEDIEPLKAALEINVEQIANIGSQDMNDAIWLRLAKRIQDAVAEAHTHEDGIVITHGTDTMEETAFFLDTVIRTDKPIILTGAMRPSTAIGADGPANLYEAEIAVATPKKDAHGVMILAVMNDTIHAARWASKTHTTAVETFQSINAGPIGYPVDASRVFIEPKKQVPPLGYSTTAPPPLAVEILAYMHSGAGSIINDILKTGKGIIVLAGVGDGNSSKEAMAALNLAVKQGVIVVRSSRTGSGFVRNNVEVNDDKNDFVVSYDLSPQKARILLQILIANGKNKLSDIQSAFEAGF